(19) United States
(12) Patent Application Publication
(10) Pub. No.: US 2004/0180430 A1
(43) Pub. Date: Sep. 16, 2004

(54) Methods of restoring telomere length and extending cell lifespan using nuclear transfer

(76) Inventors: Michael D. West, Boston, MA (US); Jose Cibelli, Holden, MA (US); Robert Lanza, Clinton, MA (US)

Correspondence Address: Attention of Joseph Bennett-Paris, MERCHANT & GOULD P.C., P.O. Box 2903, Minneapolis, MN, 55402-0903, US

(21) Appl. No.: 10/790,640

(22) Filed: Mar. 1, 2004

Related U.S. Application Data
(63) Continuation of application No. 09/527,026, filed on Mar. 16, 2000, now abandoned.
(60) Provisional application No. 60/179,486, filed on Feb. 1, 2000.
(60) Provisional application No. 60/152,340, filed on Sep. 7, 1999.

(51) Int. Cl. C12N 5/08

(52) U.S. Cl. 435/366

(57) ABSTRACT

This invention relates to methods for rejuvenating primary cells and for making primary cells of a different type having the same genotype as a primary cell of interest. Also encompassed are methods of re-cloning cloned mammals, particularly methods where the offspring of cloned mammals are designed to be genetically altered in comparison to their cloned parent. Also included are methods for activating endogenous telomerase for the purpose of extending the life span of a primary cell.

### FIELD OF INVENTION

The present invention relates to methods for rejuvenating primary cells that are nearing senescence through nuclear transfer techniques. The methods are particularly useful for rejuvenating cells which have reached senescence early due to complex genetic manipulations or harsh selection conditions, and will increase the potential of such cells to serve as donors for the generation of cloned transgenic animals. The methods of the invention also include the making of teratoma from rejuvenated cells, which contain all three germ layers and are useful for making primary cells of a different type having the same genotype as a primary cell of interest. Such newly generated primary cells have important significance in the field of tissue engineering and organ replacement therapy. Also encompassed are methods of re-cloning cloned mammals, particularly methods where the offspring of cloned mammals are designed to be genetically altered in comparison to their cloned parent.

### BACKGROUND OF THE INVENTION

The past decade has been characterized by significant advances in the science of cloning, and has witnessed the birth of a cloned sheep, i.e. “Dolly” (Roslin Bio-Med), a trio of cloned goats named “Mira” (Genzyme Transgenics), several dozen cloned cattle (ACT), numerous generations of cloned mice, cloned goats (Genzyme Transgenics using technology licensed from ACT), and very recently, five cloned pigs (Roslin Bio-Med). The technology which enables cloning has also advanced such that a mammal may now be cloned using the nucleus from an adult, differentiated cell, which scientists now know undergoes “reprogramming” when it is introduced into an enucleated oocyte. See U.S. Pat. No. 5,945,577, herein incorporated by reference.

The fact that an embryo and embryonic stem cells may be generated using the nucleus from an adult differentiated cell has exciting implications for the fields of organ, cell and tissue transplantation. For instance, embryonic stem cells generated from the nucleus of a cell taken from a patient in need of a transplant could be made, and induced to differentiate into the cell type required in the transplant. By using techniques evolving in the field of tissue engineering, tissues and organs could be designed from the cloned differentiated cells which could be used for transplantation. Because the cells and tissues used for the transplant would have the same genotype as the patient, the problems of transplant rejection and the dangers inherent in the use of immune-suppressive drugs would be avoided. Moreover, the engineered cells and tissues could be readily modified with heterologous DNA, or modified such that deleterious genes are inactivated, such that the transplanted cells and tissues are genetically corrected if necessary.

There have been recent concerns, however, regarding the genetic age of cloned cells. A recent report by Shiels et al. (Nature (1999) 399: 316), involving Dolly, the cloned sheep, suggests that nuclear transfer may not restore telomeric length, and that the terminal restriction fragment (TRF) size observed in animals cloned from embryonic, fetal and adult cells reflects the mortality of the transferred nucleus. The implications of these findings are particularly relevant for the cloning of replacement cells and tissues for human transplantation (Lanza et al. (1999a) Nature Med. 5: 975; Lanza et al. (1999b). Nature Biotechnol. 17: 1171 (1999)). Transplanted organs which undergo premature senescence could become destructive to surrounding tissue in vivo and could actually aggravate the disease which the replacement cells are intended to treat. The Shiels et al. report also raises questions as to whether cells created by nuclear transfer will undergo premature senescence and whether cloned animals generated by nuclear transfer will exhibit decreased life spans. This in turn has serious implications for the cloning and re-cloning of high quality farm animals, which, prior to the report, was considered to be advantageous over traditional breeding techniques which are dependent on the animals reaching mating age before another generation may be propagated.

Scientists have hypothesized that telomere loss is linked to the aging process for at least two decades. See Harley, “Telomere loss: mitotic clock or genetic time bomb?” Mutation Res. (1991) 256: 271-282. The hypothesis, originally called the “marginotomy theory,” is that the gradual loss of chromosomal ends, or telomeres, leads to cell cycle exit and as a consequence, cell senescence. See Olovnikov, “A theory of Marginotomy” J. Theor. Biol. (1973) 41: 181-190. The hypothesis originally arose through the prediction that DNA polymerase, because it required some sort of primer for synthesis, would be unable to replicate the ends of chromosomes. This prediction was eventually confirmed through molecular studies which showed that the mean length of terminal restriction fragments in human fibroblast chromosomes were lost in a replication dependent manner in vitro. See Harley et al. “Telomeres shorten during aging of human fibroblasts” Nature (1990) 345:458-460.

Further evidence supporting the telomerase theory relates to the enzyme telomerase. Telomerase activity in human cells was first identified in 1989. See Morin, “The human telomere terminal transferase is a ribonucleoprotein that synthesizes TTAGGG repeats” Cell (1989) 59: 521-529. Telomerase acts to build on the ends of chromosomes, restoring telomere length. Other studies have shown that, while telomerase activity is repressed during differentiation of somatic cells, telomerase is active at some stage of gametogenesis and thus maintains telomere length in germ cells between generations. Telomerase has also been shown to be active in transformed cells. See Harley (1991) for a review.

Because nuclear transfer bypasses gametogenesis, a current hypothesis with regard to cloning is that the telomeres of clones are never regenerated, and that a cloned animal is of the same “genetic age” as its parent. In fact, it has even been noted that the technology involved in cloning further reduces the length of telomeres, because cells are cultured in the laboratory for a period of time before being used for nuclear transfer. See BBC News, “Is Dolly old before her time?” Thurs., May 27, 1999. If the “telomere theory” were true, this would mean that clones have a much shorter average life span than an animal of the same age generated via sexual reproduction, and perhaps even a shorter life span than the parents from which they are generated.

Not only does the “telomere theory” have serious implications for the field of organ transplantation, but it also calls into question the extent of genetic manipulations which may be performed to somatic cells which are to be used for nuclear transfer. For instance, a major advantage of nuclear transfer technology is that somatic cells may be more readily maintained in culture and transfected with transgenes than embryonic stem cells. This property facilitates the production of animals which produce therapeutic proteins, i.e., for instance cows which express transgenes from mammary-specific promoters enabling the production of therapeutic proteins in milk.

Likewise, if cells used for nuclear transfer are not permitted to undergo a series of genetic manipulations, i.e., either consecutively in culture or consecutively through successive cloning, it will be virtually impossible to generate animals, cells and tissues with multiple genetic manipulations. The ability to perform such complex genetic manipulations may be necessary, for example, to correct genetic abnormalities in donor cells from patients having deleterious mutations before such cells are used for nuclear transfer and organ transplantation.

One hypothesis to explain why telomeres are not regenerated through the process of reprogramming the donor cell nucleus is that telomere regeneration will be dependent on the choice of donor somatic cell types. Recent studies have shown that reconstruction of telomerase activity leads to telomere elongation and immortalization of normal human fibroblasts (Bodnar et al. (1998) Science 279: 349; Vaziri and Benchimol (1998) Curr. Biol. 8: 279), whereas similar experiments using mammary epithelial cells did not result in elongation of telomeres and extended replicative life span (Kiyono et al. (1998) Nature 396: 84). Differences between cells in the ability of telomerase to extend telomeres, or in the signaling pathways activated upon adaptation to culture, were proposed to explain the differences (de Lange and DePinho (1999) Science 283: 947).

Some researchers have suggested that telomerase activity may be cell-cycle dependent. For instance, in 1996, Dionne reported the down-regulation of telomerase activity in telomerase-competent cells during quiescent periods (G phases) and hypothesized that telomerase activity may be cell-cycle dependent. See http://telomeres,virtualave.net/regulation.html. Similarly, Kruk et al. reported a higher level of telomerase in the early S phase when compared to other points in the cell cycle (Biochem. Biophys. Res. Commun. (1997) 233: 717-722). However, other researchers have reported conflicting results, and have alternatively suggested that telomerase activity correlates with growth rate, not cell cycle (Holt et al. (1996) Mol. Cell. Biol. 16(6): 2932-2939; see also Website, id., referencing Holt, 1997, and Belair, 1997). Still others have proposed that telomerase activation is mediated by other cellular activation signals, as evidenced by the upregulation of telomerase in B cells in vitro in response to CD40 antibody/antigen receptor binding and exposure to interleukin (Website, id., citing Weng, 1997; see also Hiyama et al. (1995) J. Immunol. 155 (8): 3711-3715). But despite the rising interest in telomerase and its purported role in the process of aging and cellular transformation, the regulation of telomerase activity remains poorly understood. See, e.g., Smaglik “Turning to Telomerase: As Antisense Strategies Emerge, Basic Questions Persist,” The Scientist, Jan. 18, 1999, 13(2): 8).

The ability to regulate telomerase activity could have wide-reaching effects in the medical community, and has the potential to profoundly influence many more technologies than the regeneration of telomeres in cloned animals. Having the ability to regulate telomerase will enable the treatment of many age-related and other types of disease processes. For instance, the capability to regulate telomerase could be important for improving the effectiveness of bone marrow transplants in connection with cancer chemotherapy; telomerase therapy may be useful in replacing age-worn cells in the retina of the eye or in treating the lining of blood vessels to help prevent heart attack or stroke. Moreover, the capability to regulate telomerase may permit the control of cancerous cells. Thus, a better understanding of the regulation of telomerase has the potential to lead to a wide range of treatments, in addition to securing the efficacy of cloned tissues for tissue engineering and transplants, and ensuring and even increasing the life span of cloned and non-cloned animals.

### SUMMARY OF THE INVENTION

The present invention is based on the surprising discovery, in light of the recent doubts about the genetic age of cloned mammals, that the process of nuclear transfer is capable of rejuvenating senescent or near-senescent cells and restoring both telomere length and life span. The present invention therefore enables what would not have been deemed possible in light of the recent concerns about nuclear transfer; namely, that cells may be grown in culture until they are near senescence, and may still be used to generate cloned cells, tissues and animals that have telomeres that are comparable in length, and in fact often longer, than age-matched controls. Moreover, the present invention provides, in contrast to what has been recently suggested, that generating clones of clones, i.e. “re-cloning,” is entirely feasible, and may be repeated theoretically indefinitely.

The present invention stems from the discovery that nuclear transfer techniques may be used to extend the life span of senescent or near-senescent cells by activating endogenous (cellular) telomerase activity. This provides particular advantages over recently publicized approaches for resolving the telomere loss seen in nuclear-transfer generated animals, which focus on the exogenous expression of a cloned telomerase gene to resolve telomere shortening in cloned mammals. For instance, researchers at Geron Corporation and the Roslin Institute have recently collaborated to combine Geron's cloned telomerase gene with nuclear transfer in order resolve telomere shortening in clones. See, e.g., Businees Wire, May 26, 1999. This announcement preceded the May 27th Nature report by researchers at Roslin Institute that two other sheep (after Dolly) cloned by nuclear transfer also exhibit shorter telomeres than age-matched controls. Researchers at the University of Massachusetts involved in cloning cattle also believed that transfecting donor cells with an exogenous telomerase gene might be beneficial for the lifespan of cloned animals, despite the observation that nuclear transfer seemed to rejuvenate senescent donor cells. See http://abcnews.go.com/sections/science/Daily News/clones980522.html (1998).

The present invention is advantageous over proposed methods to express telomerase from a transfected telomerase gene, in that no genetic manipulations are required to activate telomerase and regenerate telomere length in cloned cells, tissues and animals. This advantage is particularly significant given the observation that telomerase is upregulated in many types of cancer cells. Therefore, introducing an extra gene for telomerase also introduces the possibility of inducing cell transformation, and will likely require subsequent measures aimed at controlling telomerase expression from the transfected gene. A method whereby telomerase activity may be controlled using the cell's own regulatory mechanisms is therefore preferable to inserting extra copies of the telomerase gene.

Thus, encompassed in the invention are methods of rejuvenating or increasing the lifespan of primary cells using nuclear transfer. The primary cells which would benefit from the disclosed methods include any cell, e.g. a cell which is nearing senescence, either by reaching the natural limit on population doublings or as a result of harsh selection conditions for complex genetic alterations. In fact, the present methods are applicable to any primary cell of interest, e.g. a cell that has been passaged until it is near senescence, and use of such cells as donors for nuclear transfer.

The methods of the invention allow one to reprogram the nucleus of a late passage somatic cell to an embryonic state. By allowing the embryonic cell to differentiate and develop into many different cell types, one may re-isolate the primary cell of interest in a rejuvenated or “young” state. Also, since the methods of the invention entail making an embryonic stem cell which differentiates into all different cell types, any type of cell may be generated using any primary cell of interest, so long as the genome of the primary cell has not been altered as to affect cellular development. Thus, the invention provides an invaluable way to analyze the affect of the same genetic alteration in an isogenic background (i.e., a gene knock-out or expression of a heterologous gene) in different cell types in vitro.

The methods of the present invention also increase the lifespan of a desired cell, preferably a mammalian cell, and more preferably is a human cell, e.g., that is at or near senescence, by using said cell or the nucleus or chromosomes therefrom, as a nuclear transfer donor. Preferably the process will be repeated, in that cells obtained from the resultant cloned embryo will themselves be used as nuclear transfer donors. Also, the donor cells will preferably be transgenic.

The methods of the present invention further allow one to activate telomorase expression in desired cells, e.g., mammalian cells that are at or near senescence by using said cell or the nucleus or chromosomes derived therefrom unparalleled as a donor during nuclear transfer. As discussed in detail infra, this is an unparalleled discovery as the present invention may provide a means for identifying specific molecules that are involved in the aging of cells, and which regulate cell lifespan.

In view of the inventor's finding that nuclear transfer may be used to rejuvenate or increase the lifespan of mammalian cells, e.g. cells at or near senescence cells, it is no longer a concern that cloned mammals, fetuses, teratomas, or embryos, or inner cell masses or blastocysts are of the genetic age of their parents. Thus, the invention also encompasses methods of re-cloning cloned mammals, fetuses, teratomas, embryos, etc. using nuclear transfer techniques. Such re-cloning methods are particularly useful for making transgenic mammals expressing more than one heterologous gene, or having more than one gene knocked out, because such animals can be generated by cloning techniques to generate cloned and re-cloned mammals of the same genetic background. Such methods forego the need for mating or breeding, which often results in other genetic differences and may be impossible for obtaining double knockout or double transgenic mammals having altered genes which are closely linked on the genome such that they are inherited together.

### BRIEF DESCRIPTION OF THE DRAWINGS

FIG. 1. Characterization of cell senescence in NT donor cells. (A) Cells were observed by phase contrast microscopy. The donor cells displayed an increased cell size and cytoplasmic granularity (b) as compared to the early passage BFF cells (a). (B) Representative electron micrographs of BFF (a) and donor CL53 (b) cells. Note the convoluted nucleus (n) of CL53 cells. CL53 cells are larger than BFF cells, and their cytoplasm contain abundant lysosomes (arrows) and thick fibrils. Both pictures are at the same magnification. The bar represents 2 microns. Mitochondria (m). (C) Entry of early (a, BFF) and late passage (b, CL53) cells into DNA synthesis as determined by ³H-thymidine incorporation during a 30 hr incubation (V. J. Cristofalo and B. B. Sharf (1973) Exp. Cell Res. 76: 419). The cells were processed for autoradiography, and then observed microscopically and scored for labeled nuclei. At least 400 nuclei were counted to determine the percentage of labeled nuclei, following an established protocol (Cristofalo and Sharf(1973)). (D) The donor CL53 cells exhibit reduced EPC-1 mRNA levels as determined by Northern analysis. Human fibroblasts (WI-38) at early passage (Y) and late passage (O), bovine fibroblasts at early passage (Y; BFF) and late passage (O; donor CL53), and RNAs isolated from cloned calf dermal fibroblast strains are indicated. RNA was extracted from the cells after they were grown to confluence and growth-arrested in serum free medium for 3 days (P. Chomczynski and N. Sacchi (1987) Anal. Biochem 162: 156). Equal amounts of RNA were treated with glyoxal, separated by electrophoresis on agarose gels, transferred to nitrocellulose filters electrophoretically, and hybridized with the full length EPC-1 cDNA using standard conditions (D. G. Phinney, C. L. Keiper, M. K. Francis, K. Ryder (1994) Oncogene 9: 2353).

FIG. 2. Normal cows cloned from senescent somatic cells. (A) CL53-8, CL53-9, CL53-10, CL53-11 and CL53-12 (nicknamed Lily, Daffodil, Crocus, Forsythia, and Rose, respectively) at 5 months of age; and (B) CL53-1 (Persephone, insert) at 10 months of age.

FIG. 3. Ability of nuclear transfer to restore the proliferative life span of senescent donor cells. (A) The growth curve of the original BFF cell strain (•) is compared to that of cells derived from fetus (ACT99-002) (o) that was cloned from late passage BFF cells (CL53 cells). (B) The growth curve of the CL53 donor cells demonstrating that the cultures had approximately 2 population doublings remaining. (C) Late passage CL53 cells (n=97) were seeded at clonal density, and the proliferative capacity after 1 month was collated. (D) In contrast to the clones derived from late-passage cells, single cell clones from early passage BFF cultures (original) and early-passage ACT99-002 (clone) showed a capacity for extended proliferation.

FIG. 4. Telomere length analysis. (A) Nucleated blood cells. Peripheral blood samples from cloned and control animals were analyzed by flow FISH (N. Rufer, W. Dragowska, G. Thornbury, E. Roosnek, P. M. Lansdorp (1998) Nature Biotechnol. 16: 743) in two separate blinded experiments. Duplicate samples of nucleated cells (pooled granulocytes and lymphocytes) obtained after osmotic lysis of red cells using ammonium chloride were analyzed by flow FISH as described (N. Rufer et al. (1999) J. Exp. Med. 190: 157). The average telomere fluorescence of gated mononuclear cells was calculated by subtracting the mean background fluorescence from the mean fluorescence obtained with the FITC-labeled telomere probe. Note that the age-related decline in telomere fluorescence values in normal cows and the relatively long telomeres in the cloned animals. (B) Analysis of terminal restriction fragments. Genomic DNA isolated from control cells (pre-transfection BFF bovine fibroblasts), senescent CL53 cells and fibroblasts from a 7 week old cloned fetus (ACT99-002) cells obtained by NT with senescent CL53 cells. TRF analysis of DNA fragments obtained following digestion with HinfI/RsaI was performed on a 0.5% agarose gel run for 12 hours as described (Telomere Length Assay Kit, Pharmingen, San Diego, Calif.). Lane 1: controls DNA from CEPH lymphoblastoid human cell line 134105; lane 2: biotinylated markers (Pharmingen); lane 3: TeloLow control DNA (Pharmingen, mean TRF length 3.3 kb); lane 4: senescent CL53 cells; lane 5: BFF fibroblasts pre-transfection; lane 6: ACT99-002 (cloned) cells. (C) TRF analysis as in B following electrophoresis for 24 hours on a 0.5% agarose gel. Lane 1: ACT99-002 cells (mean TRF length 19.3 kb); lane 2: BFF056H fibroblasts pre transfection (mean TRF length 17.9 kb); lane 3: senescent CL53 cells (mean TRF length 16.2 kb); lane 4 TeloHigh control DNA (Pharmingen, mean TRF length 11.3 kb); lane 5: control DNA from CEPH lymphoblastoid human cell line 134105; lane 6 biotinylated lambda DNA cut with Hind III (molecular weight markers). (D) Flow FISH analysis of pre-transfection BFF bovine fibroblasts, senescent CL53 cells and ACT99-002 fibroblasts. Cells were analyzed following hybridization with or without FITC- (C₃TA₂)₃ peptide nucleic acid probe (respectively gray and black histograms). Single cells were gated on the basis of light scatter properties. Note the higher autofluorescence in the senescent CL53 cells used as nuclear donor. Fluorescence was measured on a linear scale. After subtraction of background fluorescence ACT99-002 (cloned) cells have the highest fluorescence followed by BFF (original) cell. The senescent CL53 cells appear to have the lowest specific fluorescence.

FIG. 5. Telomerase is expressed in reconstructed embryos but not in donor bovine fibroblasts. Telomerase activity was measured using a Telomeric Repeat Amplification Protocol (TRAP) assay kit (Pharmingen, San Diego, Calif.). Lysates from adult donor senescent (CL53) fibroblasts and day 7 reconstructed bovine embryos (n=15) were obtained and used in the TRAP assay. Lane 1: extract from 4000 K562 human erythroleukemia cell lie cells; lane 2: 20 bp ladder; lane 3: no cell extract; lane 4: heat treated embryo (n=1) extract; lane 5, n=10; lane 6, n=1; lane 7, n=0.1; lane 8, n=0.01); lane 9 extract from 4000 donor CL53 fibroblasts; lane 10-11 controls for fibroblast extract (resp. no TS template and heat inactivated extract); lane 12: 20 bp ladder. All lanes contain the internal control TRAP reaction (36 bp).

### DETAILED DESCRIPTION OF THE INVENTION

The present invention includes methods of rejuvenating primary cells. “Primary” cells is intended to mean that such cells are not tumorgenic or transformed, and are capable of being reprogrammed and of facilitating embryonic development after said cell or a nucleus of such a cell or chromosome from said cell is transferred to an enucleated oocyte. By “rejuvenated” the inventors mean that the possible number of population doublings remaining for said primary cell is increased, and the telomeres are increased to at least a length comparable to an age-matched control.

In a preferred embodiment of the invention, the primary cells to be used for the present invention are senescent cells, or cells that are near-senescence. However, the present methods are applicable for any desired primary cell, preferably a human cell. Replicative senescence is a physiological state distinguishable from quiescence achieved by either serum starvation or density-dependent inhibition of growth of young cells (West et al. (1989) Exp. Cell Res. 184: 138; West et al. (1996) Exp. Gerontol. 31: 175; and Pignolo et al. (1998) Exp. Gerontol. 33: 67), and appears to involve a block in late G₁ near the G₁/S boundary in the cell cycle (Cristofalo and Pignolo Exp. (1996) Gerontol. 31: 111; Gorman and Cristofalo (1986) Exp. Cell Res. 167: 87; and Cristofalo et al. (1992) Aging and Cellular Defense Mechanisms, Franceshi et al., Eds. (New York Academy of Sciences, New York), pp.187-194).

Senescent cells may be identified by a variety of means known in the art. For instance, phase contrast light microscopy, and ultrastructural analysis by electron microscopy may be used to verify features of replicative senescence, including prominent and active Golgi apparati, increased invaginated and lobed nuclei, large lysosomal bodies, and an increase in cytoplasmic microfibrils as compared to the young cells (Lipetz and Cristofalo (1972) J. Ultrastruct. Res. 39: 43). In addition, senescent cells have a reduced capacity to enter S phase as measured by a decrease in the incorporation of ³H-thymidine and a significant increase in the staining of senescence-associated β-galactosidase (G. P. Dimri et al (1995) Proc. Natl. Acad. Sci. USA 92: 9363). Senescent cells also exhibit a reduction in EPC-1 (early population doubling level cDNA-1) (Pignolo et al. (1993) J. Biol. Chem. 268: 8949) mRNA levels as compared to early passage cells, and a down-regulation of gasI gene expression as compared to quiescent cells (Cowled et al. (1994) Exp. Cell Res. 211: 197-202).

Generally, senescent cells are isolated by propagating cells until they reach a state of irreversible growth arrest. By “near-senescence” the present inventors mean that such cells have the capability to divide no more than about 3 to 6 times, but are preferably less than 2 or 3 population doublings from replicative senescence. Although the preferred means of generating senescent cells for nuclear transfer is to passage primary cells until greater than about 90 to 95% of their lifespan is completed, senescence and senescent-like states can also be induced by exposing cells to various agents, including serum mitogens and Cdk inhibitors (McConnell et al. (1998) Current Biol. 8: 351-354).

The methods of the present invention may employ cell rejuvenation to generate cloned animals, or may be used to rejuvenate a primary cell of interest for other purposes. Such methods may include:

a. transferring said primary cell, the nucleus from said primary cell, or chromosomesfrom said primary cell to a recipient oocyte or egg in order to generate an embryo;

b. obtaining an inner cell mass, embryonic disc and/or stem cell using said embryo;

c. injecting said inner cell mass, embryonic disc and/or stem cell into an immune-compromised animal to form a teratoma;

d. isolating said resulting teratoma;

e. separating the different germ layers for the purpose of identifying specific cell types;

f. isolating a cell of the same type as the primary cell.

The teratomas, inner cell masses, embryonic disc and embryonic stem cells isolated according to the invention will have telomeres that are at least as long if not longer than those of the donor primary cell, and are also an aspect of the invention. A method whereby the teratoma cells, inner mass cells, blastocyst cells or embryonic cells are then used as subsequent nuclear donors is also envisioned. Such a method is particular suitable for isolating primary cells, teratomas, ES cells, etc. having multiple transgenes or genetic alterations, and may be repeated indefinitely until the desired number of genetic changes have been accomplished.

The primary cell used for the methods of the invention may be any cell type. Suitable cells include by way of example immune cells such as B cells, T cells, dendritic cells, skin cells such as keratinocytes, epithelial cells, chondrocytes, cumulus cells, neural cells, cardiac cells, esophageal cells, primordial germ cells, cells of various organs including the liver, stomach, intestines, lung, kidneys, etc. In general, the most appropriate cells are easily propagatable in tissue culture and can be easily transfected. Preferably, cell types for transfecting heterologous DNA and performing nuclear transfer are fibroblasts.

The primary cell may be from any type of animal or mammal, such as pig, goat, cat, dog, rat, mouse, bovine, buffalo, sheep, horse, human, non-human primate, but is preferably an ungulate cell, and most preferably a bovine cell. The immune-compromised animal may be any animal capable of supporting teratoma formation, and is immune-compromised to the extent that no rejection of the developing teratoma occurs. For example, the immune-compromised animal may be a SCID or nude mouse.

The method is particularly useful for isolating primary cells having complex or compound manipulations, i.e., more than one transfected heterologous gene and/or gene knockout, where it may be difficult to keep the primary cell in culture long enough to affect all the desirable genetic alterations. Thus, the primary cell could undergo a first genetic manipulation, could then be rejuvenated according to the methods of the invention, and could then go through a second genetic manipulation once the genetic clock has been “reset.” Accordingly, a rejuvenated primary cell according to the invention may have at least one alteration to the genome depending on the complexity of the genetic manipulation and the number of times it has gone through the rejuvenation process. Rejuvenated, genetically altered cells generated by the methods of the invention are also encompassed.

The invention also includes methods of making primary cells having the same genotype as a first cell which is of a different cell type. Such a method is made possible by the process of rejuvenation, which is affected by transferring a first primary cell, the nucleus of a first primary cell, or the chromosomes from a first primary cell into an enucleated recipient oocyte to generate a teratoma, which contains the germ layers ectoderm, mesoderm and endoderm. An enucleated egg just after fertilization may also be used. Thus, virtually any type of cell may be isolated from the teratoma or by cells from the teratoma to developmentally differentiate. Specific cell markers unique to the particular cell type of interest are known in the art and may be used to identify the cloned primary cell.

In general, methods of making primary cells of a different type than the cell used for nuclear transfer comprise:

a. transferring a first cell, the nucleus from said first cell, or the chromosomes from a first cell to a recipient oocyte in order to generate an embryo;

b. obtaining an embryonic disc and/or stem cell using said embryo;

c. injecting said inner cell mass, embryonic disc and/or stem cell into an immune compromised animal to form a teratoma;

d. isolating said resulting teratoma;

e. separating the different germ layers for the purpose of identifying specific cell types;

f. isolating a cell of a different type than the first cell.

In embodiments wherein the donor cell, nucleus or chromosomes are human, the genome of the primary cell may be modified such that the cell is capable of producing a viable embryo. This may be affected by inactivating or knocking out one or more genes required for the formation of one of the three germ layers, or by expressing a “suicide” gene from a developmentally regulated promoter specifically expressed in a cell type contained in a germ layer which is not of interest. Alternatively, gene knockouts or suicide gene expression could be targeted to genes specifically required for attachment to or development in a mammalian uterus.

As discussed above, preferably the first (nuclear donor) cell is a fibroblast. The method may be formed using any species of cell, and finds particular use in human therapeutic cloning in the generation of cloned organs and tissues for transplantation. Thus, the methods may be performed using human cells, and the primary cells isolated may be used to generate a tissue (for transplantation into a patient in need of a transplant).

Preferred types of primary cells to be generated by the disclosed methods are smooth muscle, skeletal muscle, cardiac muscle, skin and kidney cells. The method may further comprise isolating cells from the teratoma and growing said cells in the presence of growth factors to facilitate further differentiation. In particular, the genome of the first cell is altered prior to nuclear transfer, such that the new primary cells and engineered tissues that are generated express at least one therapeutic protein, or fail to express a native protein that may have been detrimental to the donor patient. The cells and tissues generated by the disclosed methods are also encompassed.

Preferred applications of cells and tissues generated by the methods disclosed herein include the production of neurons, pancreatic islet cells, hepatocytes, cardiomyocytes, hematopoietic cells, and other desired differentiated cell types and tissues containing.

These cells and tissues, which optionally may be transgenic, may be used for cell, tissue and organ transplantation, e.g., treatment of burns, cancer, chronic pain, diabetes, dwarfism, epilepsy, heart disease such as myocardial infarction, hemophilic, infertility, kidney disease, liver disease, osteoarthritis, osteoporosis, stroke, affective disorders, Alzheimer's disease, enzymatic defects, Huntington's disease, hypocholesterolemine, hypoparathyroidase, immunodeficiencies, Lou Gehrig's disease, macular degeneration, multiple sclerosis, muscular dystrophy, Parkinson's disease, rheumatoid arthritis, and spinal cord injuries.

Because nuclear transfer techniques are useful in generating cloned mammals as well as cloned cells and tissues, the methods of the present invention are also useful in making cloned mammals having complex or compound genetic alterations. In particular, the invention encompasses a method of re-cloning a cloned animal, wherein said re-cloned animal has been genetically altered with respect to the cloned animal. Such a method would not have been attempted without the finding of the present invention, which reveals that nuclear transfer rejuvenates nearly-senescent cells and restores telomere length. If the re-cloned mammal was of the same genetic age as the cloned genetic mammal (which is, in turn, the same genetic age of the first nuclear donor), the feasibility of the method would decline depending on the generation of the clone. The results obtained by the present inventors to-date suggest that this is not the case and that in fact re-cloning can be effectuated as many times as desired.

In particular, a method of re-cloning according to the present invention comprises the following steps, and may be used to make a cloned animal having at least two genetic modifications:

a. obtaining a primary cell from an animal of interest,

b. making a first genetic modification to said primary cell by inserting heterologous DNA and/or deleting native DNA,

c. using said first genetically modified primary cell as a nuclear donor for nuclear transfer to an enucleated oocyte,

d. obtaining a cloned embryo, fetus or animal having said first genetic modification,

e. obtaining a cloned primary cell from said cloned embryo, fetus or animal,

f. making a second genetic modification to said cloned primary cell by inserting heterologous DNA and/or deleting native DNA,

g. using said cloned primary cell having said first and second genetic modifications as a nuclear donor for nuclear transfer to an enucleated oocyte,

h. obtaining a re-cloned embryo, fetus or animal having said first and second genetic modifications.

This process can be repeated as many times as desired, where at least one recloning step utilizes a donor cell that has been propagated to senescence or near-senescence such that the telomeres of the reclones cell are regenerated upon nuclear transfer. In particular, the method of the invention further comprises steps where said re-cloned embryo, fetus or animal is again re-cloned, and wherein a third genetic modification is made such that the further re-clone has the first, second and third genetic modifications. Accordingly, the method may be used to generate animals having numerous genes knocked out, inserted or substituted, and may be used to generate animals having entire cell systems replaced or modified, i.e., substitution of the human immunological system for that of the bovine, substitution of genes involved in complex enzymatic pathways such as those involving the clotting factors, or the complement cascade, etc.

The method of re-cloning of the present invention will allow the creation of complex animal models for the study of diseases which involve multiple genes and or cell types, and may not be able to be duplicated by the typical animal model which expresses a single transgene, or has a single gene of interest knocked out. Moreover, such animal models may be used to study the effect of therapeutic genes in a particular complex genetic background. Such animal models may also be used to produce and test products that regulate the expression of different genes, to knock out genes that are involved in eliciting immune responses, to substitute collagen genes or other structural proteins genes with homologous counterparts, etc.

The present invention involves the surprising discovery that senescent cells may be rejuvenated, telomorase may be activated and that telomeres may be regenerated, by the process of nuclear transfer. Thus, the present invention involves the discovery of a new way to activate telomerase activity, which has applications far beyond that of extending telomeres and replicative lifespan. In particular, the invention provides a method for isolating the mechanism(s) of telomerase activation, as well as a means of regulating telomerase activity using the identified mechanisms.

For instance, the cytoplasm of an oocyte can be fractionated and the fractions placed in association with a mortal cell, or a mortal cell nucleus, or telomeres, to assay for telomerase activation and telomere extension. Through such an assay, the activity in oocytes responsible for reactivating telomerase can be identified and isolated. Similarly, RNA or cDNAs can be isolated from the oocyte and transfected into a mortal cell, or expressed in a cell-free system for detecting telomerase activity, and transfected cells or cell-free systems demonstrating telomerase activity may be identified. Such methods could be supplemented with subtractive hybridization techniques in order to enrich for RNAs which are expressed during embryogenesis and not during senescence. In this way, genes encoding enzymes potentially involved in telomerase activation may be identified.

Oocytes or eggs in the period just following fertilization may contain more than one gene or protein involved in telomerase activation. While not wishing to be held to any specific theory, the present inventors believe that there exists at least one regulatory protein or RNA in oocytes, or in ES cells or germ cells resulting from the development of oocytes that are nucleated by nuclear transfer, that is involved in the regulation of telomerase activity, and responds particularly to some aspect of the senescence cellular environment. It is possible that such protein(s) or RNA(s) activate telomerase or telomerase gene expression directly, but it is also possible that such proteins or RNAs work by inhibiting a suppressor of telomerase that exists or is expressed in senescent or near-senescent cells.

For instance, Xu et al. demonstrated that re-expression of the retinoblastoma protein in tumor cells induces senescence and inhibits telomerase activity (Oncogene (1997) 15: 2589-2596). A recent report also suggests that a gene on chromosome 3 may be involved in transcriptional repression of hTERT, the catalytic subunit of telomerase. See http://claim.springer.de/EncRef/CancerResearch/samples/0001.htm. Several proteins have also been identified that interact directly with telomerase, such as p23/hsp90 (molecular chaperones) and TEP1 (telomerase associated protein 1). Id. Researchers at Lawrence Berkeley National Laboratory have purported cloned two additional human telomere-associated proteins (Tin 1 and Tin 2). Federal Technology Report, Dec. 30, 1999, Partnership Digest, Technology Watch, p. 9. Thus, the regulatory mechanism identified by the present methods could operate by binding to or inhibiting the expression of a telomerase binding protein or a telomerase repressor, consequently increasing telomerase activity, but could also regulate telomerase activity by upregulating gene expression or enhancing protein stability.

The present invention includes methods of identifying at least one gene that either directly or indirectly enhances telomerase activity. Such methods could involve screening a cDNA or mRNA library generated from an embryo or embryonic stem cell for members that enhance telomerase activity in a senescent or near-senescent cell. The methods may also involve identifying at least one gene that either directly or indirectly suppresses telomerase activity, comprising, screening a cDNA or mRNA library generated from a senescent or near-senescent cell for members that suppress telomerase activity in an embryonic stem cell. Telomerase activity may be measured by any one of several methods known in the art, including measurement of reporter gene expression, e.g., a hTRT gene or protein fusion. A preferred reporter molecule is green flourescent protein (GFP). Telomerase activity may also be measured using the TRAPeze assay. Screening methods may be combined with other known methods for the purpose of increasing the effectiveness of the screening procedure, for instance, by subjecting cDNA or mRNA libraries to subtractive hybridization with a cDNA or mRNA library from a senescent cell prior to library screening if the test library is generated from an oocyte or an ES cell, or vice versa.

The present invention also encompasses methods of identifying a protein that enhances telomerase activity, comprising (a) collecting fractions from the cytoplasm of an oocyte or embronic stem cell, (b) adding them to a cell-free system designed from a senescent or near-senescent cell, and (c) measuring for changes in telomerase activity that result from exposure to specific oocyte or ES cell cytoplasmic fractions. Methods for screening for compounds that inhibit telomerase activity are also included, and would comprise exposing an embryonic stem cell generated by nuclear transfer techniques using a senescent or near-senescent donor cell to a compound to determine whether said compound inhibits telomerase activity.

The present invention also includes the regulatory compounds, proteins and nucleic acids identified by the methods described above and pharmaceutical compositions comprising the same, which may be isolated and employed as exogenous telomerase activating agents according to the methods and purposes described herein, i.e., for the treatment of age-related diseases, the treatment of aged tissues such as retinal cells, the therapy of cancer, and the improving the effectiveness of bone marrow transplants.

The scope and spirit of the present invention are illustrated by the way of the disclosed examples.

### EXAMPLE 1

### Fetal Donor Cells

This preliminary experiment suggested that somatic cell nuclear transfer can be used to restore the life-span of primary cultured cells. When fibroblasts from a 6 week-old fetus were cultured to senescence, they underwent approximately 30 population doublings, with an average cell cycle length of 28 to 30 hours. To test whether these cells could be rescued from senescence by nuclear transfer, a 40-day old fetus was generated using cells within 0.8 populations doublings from senescence. Fibroblasts derived from this fetus underwent 31 population doublings, as compared to 33 doublings for fibroblasts from a same-age fetus conceived normally. This data suggested that nuclear transfer is capable of rejuvenating senescent cells.

### EXAMPLE 2

### Cloned Calves Derived From Senescent Donor Somatic Cells

A somatic cell strain was derived from a 45-day-old female bovine fetus (BFF) and transfected with a PGK driven selection cassette. Cells were selected with G418 for 10 days, and five neomycin resistant colonies were isolated and analyzed for stable transfection by Southern blotting using a full length cDNA probe. One cell strain (CL53) was identified as 63% [total nuclei] positive for the transgene by FISH analysis, and was chosen for the nuclear transfer studies described in this study.

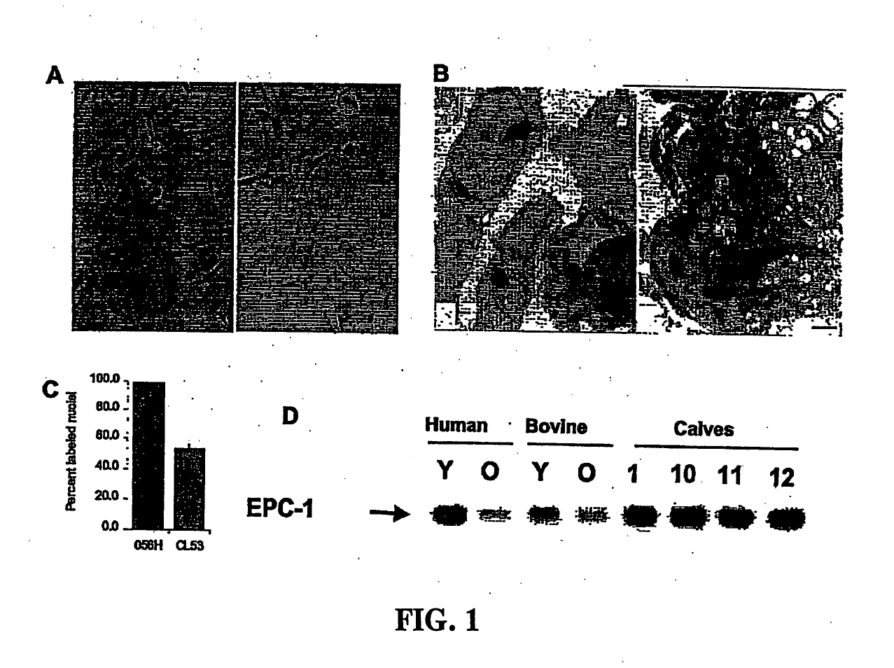

The CL53 fibroblast cells, which were characterized as negative for cytokeratin and positive for vimentin, were passaged until greater than 95% of their lifespan was completed. The morphology of the cells was consistent with cells close to the end of their lifespan as indicated by the phase contrast pictures of the cells by light microscopy (FIG. 1A). A more detailed ultrastructural analysis by electron microscopy demonstrated that these cells exhibited additional features of replicative senescence, including prominent and active Golgi apparati, increased invaginated and lobed nuclei, large lysosomal bodies, and an increase in cytoplasmic microfibrils as compared to the young cells (FIG. 1B) (27). In addition, these late passage cells exhibited a senescent phenotype in showing a reduced capacity to enter S phase as measured by a decrease in the incorporation of ³H-thymidine (FIG. 1C) and a significant increase in the staining of senescence-associated β-galactosidase (SA-β-gal; data not shown) (28). Furthermore, these cells exhibit a reduction in EPC-1 (early population doubling level cDNA-1)(29) mRNA levels as compared to early passage bovine BFF cells in a manner analogous to the changes observed during the aging of WI-38 cells (FIG. 1D).

A total of 1896 bovine oocytes were reconstructed by nuclear transfer using senescent CL53 cells as previously described (13). Eighty-seven blastocysts (5%) were identified after a week in culture. The majority of the embryos (n=79) were transferred into progestin-synchronized recipients, and 17 of the 32 recipients (53%) were detected pregnant by ultrasound 40 days after transfer. One fetus was electively removed at week 7 of gestation (ACT99-002), whereas 9 of the remaining recipients (29%) remained pregnant by 12 weeks of gestation. Three of these cows aborted at days 252 (twins), 253, and 278 of gestation. The remaining six recipients continued development to term. The rates of blastocyst formation (5%), and early (53%) and term (19%) pregnancies using senescent CL53 cells were comparable to those of control embryos produced using non-senescent donor (CL57) cells obtained from early passage BFF cells (5%, 45%, and 13%, respectively).

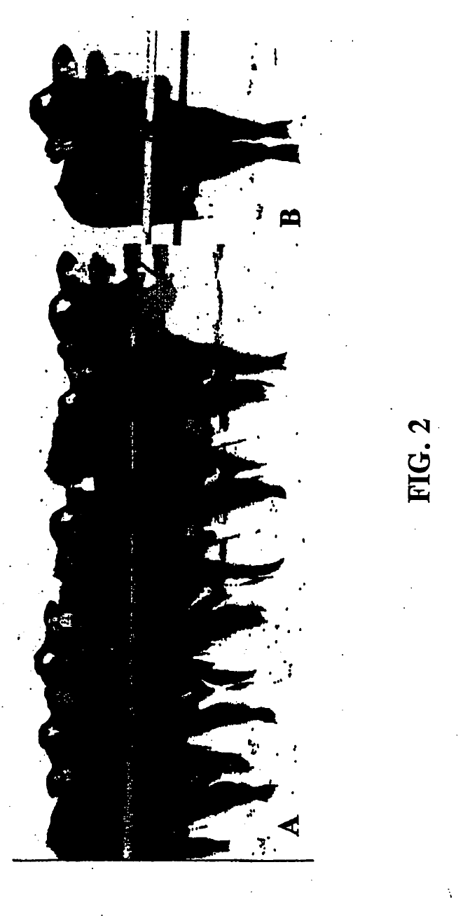

Calves CL53-1, CL53-8, CL53-9, CL53-10, CL53-11, and CL53-12 were delivered by elective cesarean section at 280, 273, 273, 273, 266, and 266 days of gestation, respectively (FIG. 2). Genomic analyses confirmed the presence of the transgene in two of the animals (CL53-1 and CL53-12), as well as the fetus that was removed electively at day 49 of gestation. At birth, the presentation of the cloned calves was consistent with previous published reports (13, 15, 30,31). In general, birth weights (51.6±3.6 kg) were increased and several of the calves experienced pulmonary hypertension and respiratory distress at birth as well as incidence of fever after vaccinations at 4 months. Following the first 24 hours, the calves have been vigorous with minimal health problems. However, we have noted a moderate incidence of polyuria/polydypsia and lowered dry matter intake during the first two months. The occurrence of these complications was linked neither to the donor cell population (isolate 53 or 57) nor the presence or absence of transgene integration. After approximately 2 months all of the calves have performed well and resemble healthy control calves generated from both in vitro fertilization and in vivo embryo transfers. All six of the cloned animals remain alive and normal 5 to 10 months after birth.

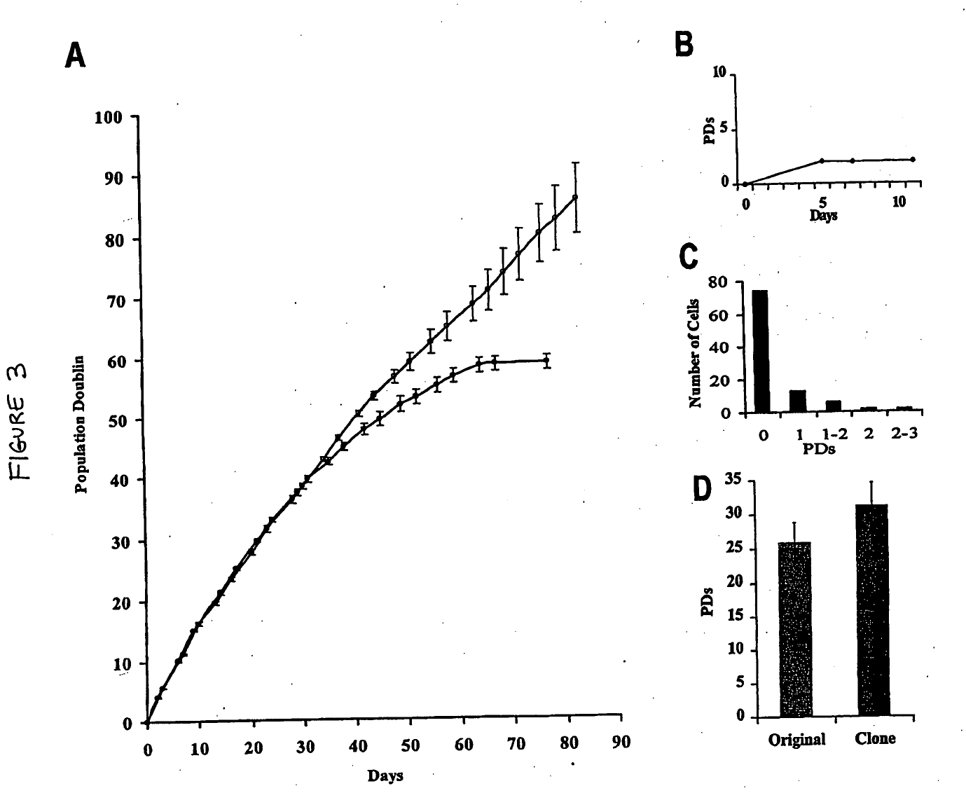

Dermal fibroblasts were isolated from the cloned calves, and mRNA prepared as described in FIG. 1D. The cells expressed EPC-1 mRNA levels comparable or higher than the early passage fetal cells. To exclude the possibility that there was a small proportion of nonsenescent cells that gave rise to the cloned animals, CL53 donor cells were seeded at both normal and clonal densities. As shown in FIG. 3B, the cells were 2.01±0.11 (SEM) population doublings from replicative senescence. Less than 12% (11/97) and 3% (2/97) of cells seeded at clonal density underwent more than 1 or 2 population doublings, respectively, whereas none of the cells divided more than 3 times (FIG. 3C). In contrast, early passage (pre-transfection) BFF cells underwent 47.8±0.9 population doublings, with an average cell cycle length of 17.8±0.7 hours during the logarithmic growth phase (FIG. 3A).

To test whether the somatic cell NT procedure restored the proliferative life span of the senescent donor cells, we cultured fibroblasts from an electively removed 7-week-old fetus (ACT99-002). Cell strains from it underwent 85.3±5.6 population doublings, with a cell cycle length of 17.7±0.8 hours during the logarithmic growth phase (FIG. 3A). One-cell clones (n=5) were generated from the cloned (ACT99-002) and original (BFF) age-matched fetuses, and cultures characterized as fibroblasts by immunohistochemical staining were isolated. These one-cell clones underwent 31.2±3.4 and 25.9±2.9 population doublings from the cloned and original fetuses, respectively (FIG. 3D). These data suggest that cloning is capable of resetting the life span of senescent cells, and that the cellular age of the fetus does not reflect the number of times the donor cells doubled in culture before NT.

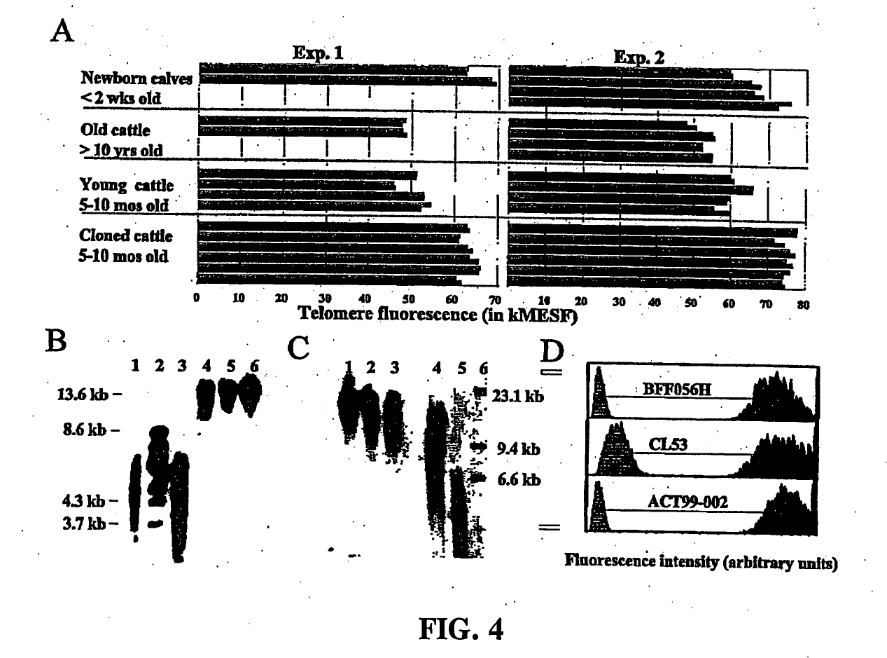

To further investigate the ability of NT to rescue senescent cells, the telomere lengths in nucleated blood cells of the cloned animals were compared to age-matched control animals, newborn calves (<2 weeks old) and old cows (10 to 19 years old) using flow cytometric analysis following in situ hybridization with directly FITC-labeled (CCCTAA) peptide nucleic acid probe (flow FISH) (32, 33). The results of two separate experiments (FIG. 4A) are indicative of complete restoration of telomere length (63.4±1.7 vs. 51.0±3.1 kMESF [mean±s.d., P<0.0001, exp. 1], and 75.7±1.7 vs. 61.4±3.2 kMESF [P<0.0001, exp. 2] in cloned animals relative to age-matched controls. Indeed, the telomeres of the clones animals were statistically longer than the four newborn calves (exp. 2) (75.3±1.2 vs. 66.9±1.4, P<0.0002). The mean telomere lengths of the old cattle were 47.7±0.7 kMESF and 52.0±3.6 kMESF for experiments 1 and 2, respectively.

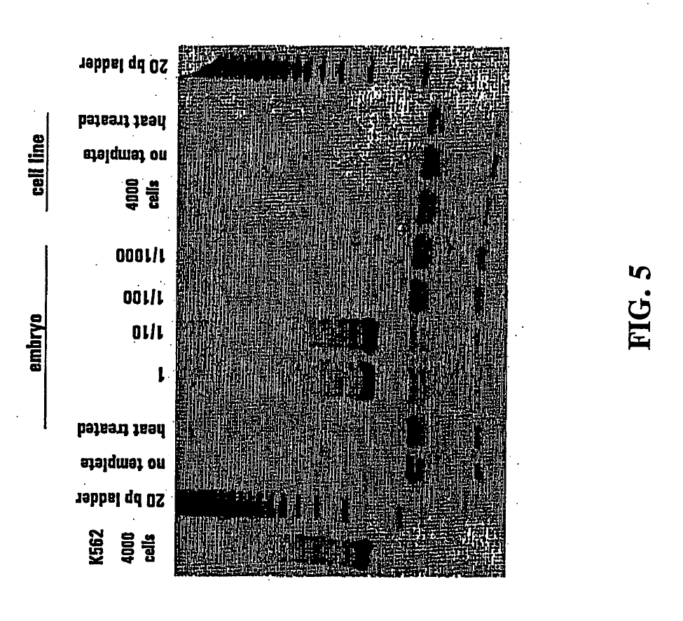

Telomere length dynamics was also studied in the senescent (CL53), control (pre-transfection BFF) and cloned (ACT99-002) cells using Southern analysis of terminal restriction fragments (34). The results (FIG. 4B-D) were consistent with the flow FISH analysis of the nucleated blood cells. The telomeres were longer in the cells derived from the cloned embryo (19.3 kb) than in the senescent and early-passage donor cells (16.2 and 17.9 kb, respectively) (compare lanes 4, 5 and 6, FIG. 4B). These results were confirmed by flow cytometric analysis of telomere length (flow FISH, ref 32) of the same cells (FIG. 4D). High levels of telomerase activity were also detected in reconstructed day 7 embryos tested by the TRAP assay (FIG. 5, lanes 5-8), whereas the bovine fibroblasts used as donor cells in the nuclear transfer experiments were negative (FIG. 5, lane 9).

Discussion

Telomere restoration has not been previously described in cloned animals. Our results differ markedly from the study by Shiels et al. (20), in which telomere erosion did not appear to be repaired after nuclear transfer in sheep. The telomere lengths of three cloned animals 6LL3 (Dolly, obtained from an adult donor cell), 6LL6 (derived from an embryonic donor cell) and 6LL7 (derived from a fetal donor cell) were found to be decreased relative to age-matched control animals. The authors suggested that full restoration of telomere length did not occur because these animals were generated without germline involvement. They further suggested that the shorter TRF in Dolly was consistent the time the donor cells spent in culture before nuclear transfer. The present findings are significant, not only because viable offspring were produced from senescent somatic cells, but because the nuclear transfer procedure appeared to extend the telomeres of the animals beyond that of newborn and age-matched control animals. It is not known whether the longevity of these animals will be reflected by the telomeric measurements, although cells derived from a cloned fetus were observed to have a longer proliferative life span than those obtained from the original same-age nonmanipulated fetus. Indeed, the mean TRF size observed in the later cells was in agreement with these findings.

In discussions about cloning, it is commonly asked whether the animals generated by nuclear transfer are the result of the use of some rare cell rather than the majority of the cells in the culture. Mass cultures have multiple lineage's with various maximum achievable cell lifespans (43). Indeed, the late passage cells used in the present study represent cells that originally had the greatest lifespan. If there were a subset of young cells with 20 or more population doublings remaining in the late passage culture, they would have out-proliferated the culture as is seen in mouse cell culture where spontaneous immortalization is common. In anticipation of this objection, we plated the donor cells at clonal densities and scored the proliferative lifespan of every cell. Three-hundred and thirty-nine of the 347 cells (98%) underwent less than 3 PDs, whereas 347/347 (100%) underwent 4 or less PDs. Furthermore, the cells were grown in high serum (15%) concentrations, and young cells would have been rapidly proliferating and easily observed in the dish. The probability of a young cell in out sample is therefore <1/347. Seven animals (6 term animals and 1 fetus) were nevertheless cloned from the population of senescent fetal cells. It is therefore highly improbable that we, by chance, cloned the animals from undetectable young cells (P<0.001, Chi-square).

The differences between this study and that reported by Shiels et al. (20) could be due to differences in the choice of donor somatic cell types. Wilmut et al. (12), for instance, used quiescent (G_{O}) donor mammary epithelial cells to produce Dolly, whereas senescent (G₁) fibroblasts were used in the present experiments. Indeed, recent studies have shown that reconstruction of telomerase activity leads to telomere elongation and immortalization of normal human fibroblasts (35,36), whereas similar experiments using mammary epithelial cells did not result in elongation of telomeres and extended replicative life span (37). Differences between cells in the ability of telomerase to extend telomeres, or in the signaling pathways activated upon adaptation to culture, were proposed to explain the differences (38). Other investigators, however, report that the exogenous expression of hTERT extends telomeres and immortalizes human mammary epithelial cells (J. Shay, personal communication).

Previous studies have documented significant up-regulation of telomerase activity during early bovine embryogenesis (39). The elongation of telomeres in the present study suggests that reconstructed bovine embryos contain a mechanism for telomere length regeneration and maintenance, providing chromosomal stability throughout the events of pre- and post-attachment development. The ability of nuclear transfer to restore somatic cells to a phenotypically youthful state may have important implications for agriculture and medicine.

### EXAMPLE 3

### Nuclear Transfer Using Adult Donor Cells

The above data obtained with fetal fibroblast donors are consistent with experiments performed using senescent cells obtained from adult animals. Dermal fibroblasts were grown from three Holstein steers. Single cell clones were isolated and population doublings counted until senescence. Nuclear transfer was performed using these fibroblast cells that were at or near senescence. Fetuses were removed from the uterus at week 6 of gestation and fibroblasts isolated from them and cultured until senescence. Cells were analyzed by imunohistochemistry and were shown to be fibroblasts. The number of population doublings in the original cells from the adult animals at the time of nuclear transfer (counted as number of PDs before senescence) and from 6-week-old fetuses generated from them are shown in Table 1. Cell strains isolated from the cloned fetuses underwent an average of 89.4±0.9 PDs as compared to 60.5±1.7 PDs for cell strains generated from normal age-matched (6-week-old) control fetuses (P<0.0001). These data suggest that cloning is capable of resetting (and indeed, extending) the life span of somatic cells, and that the cellular age of the fetus does not reflect the number of times the donor cells doubled in culture before NT.

TABLE 1
Population doublings in fibroblasts derived from
normal fetuses and fetuses generated from
clonal populations of adult senescent cells

| | PDs left at time of nuclear | |
|---|---|---|
| | transfer in original adult | PDs in fibroblasts isolated |
| | cells | from the fetus |
| Cloned Fetus | | |
| 25-1 | 0.26 | 90.14 |
| 25-2 | 0.0 | 91.44 |
| 14-1 | 4.0 | 89.27 |
| 14-2 | 1.0 | 90.34 |
| 22-1 | 2.5 | 85.86 |
| Normal fetus | | |
| 1-1 | — | 59.64 |
| 2-1 | — | 67.37 |
| 3-1 | — | 60.18 |
| 3-2 | — | 59.82 |
| 3-3 | — | 55.66 |

### EXAMPLE 4

### Analysis of Adult Donor Cell Types

Tissue biopsies will be obtained from all three germ layers from an adult cow (obtained at time of slaughter). In particular at least the following cells will be collected:

ectoderm—keratinocytes

mesoderm—dermal fibroblasts

endoderm—gut epithelium

A portion of the above three cell types will immediately be evaluated to determine telomere length. This can be affected by various methods. The remaining portion of all three cell types will be cultured until senescence. During culturing, a portion of each population will be retained and frozen. The different frozen cell samples will be labeled based on their particular population doubling.

Thereafter, the telomere length for the various cell samples will be evaluated, including especially the cells obtained at the time of senescence.

### EXAMPLE 5

### Cloned Calves Generated From Adult Senescent Donor Somatic Cells

The cells obtained from Example 4 will be used to obtain cloned bovine fetuses. In particular, bovine clones will be produced using all 3 cell types, and using cells from different population doublings, i.e., from 0.8 population doublings away from senescence. The cloned bovine fetuses will be produced substantially according to the methods disclosed in U.S. Pat. No. 5,945,577, incorporated by reference herein. The cloned fetuses will be removed at 40 days and cells of all three types isolated therefrom, e.g., keratinocytes, dermal fibroblasts, and gut epithelial cells.

Additionally, as a control, two same-age (40 day) wild-type fetuses will also be used to recover the same three types of cells. These cells, as well as those isolated from the cloned fetuses, will be cultured until senescence.

Again, telomere length of these different types of cultured cells will be determined immediately upon isolation from the animal or from such cells which are frozen upon isolation. Further, cells will again be removed and frozen from different cell populations until senescence. Thereafter, telomere length will be computed for the different cell types obtained at different cell population doublings, for cultured cells derived from cloned and wild-type embryos.

The results will be compared to the results of Example 4. These experiments are currently ongoing.

References

1. L. Hayflick, Exp. Cell Res. 37, 614 (1965).

2. L. Hayflick and P. S. Moorhead, Exp. Cell Res. 25, 585 (1961).

3. V. J. Cristofalo and R. J. Pignolo, Physiol Rev 73, 617 (1993).

4. M. D. West, O. Pereira-Smith, J. R. Smith, Exp. Cell Res. 184, 138 (1989).

5. M. D. West, Arch. Dermatol. 130, 87 (1994).

6. C. B. Harley, A. B. Futcher, C. W. Greider, Nature 345, 458 (1990).

7. R. C. Allsopp and C. B. Harley, Exp Cell Res 219, 130 (1995).

8. H. Vaziri et al, EMBO J. 16, 6018 (1997).

9. R. C. Allsopp et al, Proc. Natl. Acad. Sci. 89, 10114 (1992).

10. M. Z. Levy, R. C. Allsopp, A. B. Futcher, C. W. Greider, C. B. Harley, J. Mol. Biol. 225,951 (1992).

11. M. D. West, J. W. Shay, W. E. Wright, M. H. K. Linskens, M. H. K, Exp. Gerontol. 31, 175 (1996).

12. I. Wilmut, A. E. Schnieke, J. McWhir, A. J. Kind, K. H. S. Campbell, Nature 385, 810 (1997).

13. J. B. Cibelli et al, Science 280, 1256 (1998).

14. K. H. S. Campbell, J. McWhir, W. A. Ritchie, I Wilmut, Nature 380, 64 (1996).

15. Y. Kato et al, Science 262, 2095 (1998).

16. T. Wakayama, A. C. F. Perry, M. Zuccotti, K. R. Johnson, R. Yanagimachi, Nature 394, 369 (1998).

17. A. Baguisi et al, Nature Biotech 17, 456 (1999).

18. L. Meng, J. J. Ely, R. L. Stouffer, D. P. Wolf, Biol. Reprod 57, 454 (1997).

19. K. R. Bondioli, Mol. Reprod. Dev 36, 274 (1993).

20. P. G. Shiels et al, Nature 399 , 316 (1999).

21. R. P. Lanza, J. B. Cibelli, M. D. West, Nature Medicine 5, 975 (1999).

22. R. P. Lanza, J. B. Cibelli, M. D. West, Nature Biotechnology 17, 1171 (1999).

23. R. J. Pignolo, B. G. Martin, J. H. Horton, A. N. Kalbach, V. J. Cristofalo, Exp. Gerontol. 33, 67 (1998).

24. V. J. Cristofalo and R. J. Pignolo, Exp. Gerontol. 31, 111 (1996).

25. S. D. Gorman and V. J. Cristofalo, Exp. Cell Res. 167, 87 (1986).

26. V. J. Cristofalo, R. J. Pignolo, M. O. Rotenberg, Aging and Cellular Defense Mechanisms, C. Franceshi, G. Crepaldi, V. J. Cristofalo, J. Vijg, Eds. (New York Academy of Sciences, New York, 1992), pp.187-194.

27. J. Lipetz and V. J. Cristofalo, J. Ultrastruct. Res. 39, 43 (1972).

28. G. P. Dimri et al, Proc. Natl. Acad. Sci. USA 92, 9363 (1995).

29. R. J. Pignolo, V. J. Cristofalo, M. O. Rotenberg, J. Biol. Chem. 268, 8949 (1993).

30. J. R. Hill et al. Theriogenology 51, 1451 (1999).

31. J. P. Renard et al. Lancet 353, 1489 (1999)

32. N. Rufer et al. J. Exp. Med. 190, 157 (1999).

33. N. Rufer, W. Dragowska, G. Thornbury, E. Roosnek, P. M. Lansdorp, Nature Biotechnol. 16, 743 (1998).

34. C. B. Harley, A. B. Futcher, C. W. Greider, Nature 345, 458 (1990).

35. A. G. Bodnar et al, Science 279, 349 (1998).

36. H. Vaziri and S. Benchimol, Curr. Biol. 8, 279 (1998).

37. T. Kiyono et al, Nature 396, 84 (1998).

38. T. de Lange and R. A. DePinho, Science 283, 947 (1999).

39. D. H. Betts and W. A. King, Dev. Genetics 25, 397 (1999).

40. V. J. Cristofalo and B. B. Sharf, Exp. Cell Res. 76, 419 (1973).

41. P. Chomczynski and N. Sacchi, Anal. Biochem 162, 156 (1987).

42. D. G. Phinney, C. L. Keiper, M. K. Francis, K. Ryder, Oncogene 9, 2353 (1994).

43. J. R. Smith, R. G. Whitney, Science 207, 82-84 (1979).

What is claimed:

1. A method of rejuvenating a primary cell, comprising:
a. transferring a primary cell, the nucleus from said primary cell or chromosomes from a primary cell to a recipient oocyte or egg in order to generate an embryo;
b. obtaining an inner cell mass, embryonic disc and/or stem cell using said embryo;
c. injecting said inner cell mass, embryonic disc and/or stem cell into an immune-compromised animal to form a teratoma;
d. isolating said resulting teratoma;
e. separating the different germ layers for the purpose of identifying specific cell types;
f. isolating a cell of the same type as the primary cell.

2. The method of claim 1, wherein said primary cell is a senescent cell or a cell that is near senescence.

3. The method of claim 1, wherein said cell isolated from said nuclear transfer teratoma has telomeres that are on average at least as long as those of cells from a same age control teratoma that is not generated by nuclear transfer techniques.

4. The method of claim 4, wherein said telomeres are on average longer than those of cells from a same age control teratoma that is not generated by nuclear transfer techniques.

5. The method of claim 2, wherein said primary cell is a fibroblast.

6. The method of claim 1, wherein said immune-compromised animal is a SCID or nude mouse.

7. The method of claim 1, wherein said primary cell has at least one alteration to the genome.

8. A method of making a primary cell having the same genotype as a first cell which is of a different cell type, comprising:
a. transferring the nucleus from said first cell to a recipient oocyte in order to generate an embryo;
b. obtaining an inner cell mass, embryonic disc and/or stem cell using said embryo;
c. injecting said inner cell mass, embryonic disc and/or stem cell into an immune compromised animal to form a teratoma;
d. isolating said resulting teratoma;
e. separating the different germ layers for the purpose of identifying specific cell types;
f. isolating a cell of a different type than the first cell,
wherein the telomeres of said new primary cell are at least as long the telomeres of a same age control cell in a teratoma not generated by nuclear transfer techniques.

9. The method of claim 8, wherein said first cell is a senescent cell or a cell that is near senescence.

10. The method of claim 9, wherein said first cell is a fibroblast.

11. The method of claim 8, wherein said primary cell is of a type selected from the group consisting of smooth muscle, skeletal muscle, cardiac muscle, skin and kidney.

12. The method of claim 8, further comprising growing said cell of a different type in the presence of growth factors to facilitate further differentiation.

13. The method of claim 11, wherein said primary cell is used to generate a tissue (for transplantation into a patient in need of a transplant).

14. The method of claim 8, wherein the genome of the first cell is altered prior to nuclear transfer.

15. The cell isolated by the method of claim 8.

16. The tissue isolated by the method of claim 13.

17. The method of claim 7, wherein said genetic alteration comprises the transfection of at least one heterologous gene.

18. The method of claim 7, wherein said genetic alteration comprises the disruption of at least one native gene.

19. The method of claim 14, wherein said genetic alteration comprises the transfection of at least one heterologous gene.

20. The method of claim 14, wherein said genetic alteration comprises the disruption of at least one native gene.

21. A method of performing compound genetic manipulations in a primary cell, comprising rejuvenating said primary cell between genetic manipulations using nuclear transfer into a recipient oocyte, wherein said cell is passaged to a senescent or near-senescent state prior to nuclear transfer.

22. A method of performing compound genetic manipulations in a primary cell, comprising rejuvenating said primary cell between genetic manipulations using nuclear transfer into a recipient oocyte, wherein said cell is induced into a senescent-like or near-senescent-like state prior to nuclear transfer.

23. The method of claim 21, whereby rejuvenation results in an embryonic cell that has telomeres at least as long on average as a same age control embryonic cell.

24. A primary cell that has been genetically altered according to the method of claim 21.

25. A method of making a genetically altered animal having the same genotype as the cell of claim 24, comprising
a. transferring the nucleus of said cell into a recipient oocyte,
b. generating an embryo or embryonic stem cell from said nucleated oocyte,
c. introducing said embryo or embryonic stem cell into a recipient female, and
d. allowing said embryo or embryonic stem cell to fully develop such that said female delivers a newborn animal having the same genotype as said primary cell.

26. The genetically altered animal produced by the method of claim 25, whereby said animal has telomeres that are at least as long on average as a same age control animal.

27. A method of re-cloning a cloned animal using nuclear transfer techniques, wherein the donor cell used to supply the nucleus of the re-clone is a cell that is senescent or near senescence.

28. The method of claim 25, wherein said re-cloned animal has been genetically altered with respect to the cloned animal.

29. A method of making a re-cloned inner cell mass, blastocyst, teratoma embryo, fetus or animal containing at least two genetic modifications, comprising:
a. obtaining a primary cell from an animal of interest,
b. making a first genetic modification to said primary cell by inserting heterologous DNA and/or deleting native DNA,
c. allowing said genetically modified primary cell to multiply to senescence or near-senescence,
d. using a first genetically modified senescent or near-senescent cell as a nuclear donor for nuclear transfer to an enucleated oocyte or an enucleated fertilized egg,
e. obtaining a cloned inner cell mass, blastocyst, teratoma, embryo, fetus or animal having said first genetic modification,
f. obtaining a cloned primary cell from said cloned inner cell mass, blastocyst, teratoma, embryo, fetus or animal,
g. making a second genetic modification to said cloned primary cell by inserting heterologous DNA and/or deleting native DNA,
h. allowing said second cloned primary cell to multiply until senescence or near senescence,
i. using a senescent or near-senescent cloned primary cell having said first and second genetic modifications as a nuclear donor for nuclear transfer to an enucleated oocyte or an enucleated fertilized egg, and
j. obtaining a re-cloned inner cell mass, blastocyst, teratoma, embryo, fetus or animal having said first and second genetic modifications.

30. The method of claim 29 further comprising steps where said re-cloned inner cell mass, blastocyst, teratoma, embryo, fetus or animal is again re-cloned, and wherein a third genetic modification is made such that the further re-clone has the first, second and third genetic modifications.

31. The method of claim 30, wherein said further re-clone is generated by nuclear transfer techniques using a senescent or near-senescent donor cell.

32. The method of claim 29, wherein said re-clone has telomeres that are at least as long on average as a same age control animal that was not generated using nuclear transfer techniques.

33. The method of claim 31, wherein said further re-clone has telomeres that are at least as long on average as a same age control animal that was not generated using nuclear transfer techniques.

34. The method of claim 29, wherein the genetic modifications involve genes that are responsible for immunological function.

35. The method of claim 29, wherein said animal of interest is an ungulate.

36. The method of claim 35, wherein said animal of interest is a bovine.

37. A method of re-setting the lifespan of senescent or near-senescent cells, comprising transferring the nucleus of said cell into a recipient oocyte.

38. The method of claim 37 wherein said recipient oocyte is of a different species than said senescent or near-senescent cell.

39. The method of claim 37 further comprising generating an embryo or embryonic stem cell from said nucleated oocyte.

40. A method of identifying at least one gene that either directly or indirectly enhances telomerase activity, comprising, screening a cDNA or mRNA library generated from an embryo or embryonic stem cell for members that enhance telomerase activity in a senescent or near-senescent cell.

41. The method of claim 40 whereby enhancement in telomerase activity is measured by measuring for enhanced expression of a telomerase reporter gene.

42. The method of claim 41 wherein said telomerase reporter gene is a construct comprising the hTRT gene fused to a reporter gene.

43. The method of claim 42 wherein the construct comprises a gene fusion.

44. The method of claim 42 wherein the construct comprises a protein fusion.

45. The method of claim 40 whereby enhanced telomerase activity is measured via the TRAPeze assay.

46. The method of claim 40 whereby said cDNA or mRNA library is subjected to subtractive hybridization with a cDNA or mRNA library from a senescent cell prior to library screening.

47. A method of identifying at least one gene that either directly or indirectly suppresses telomerase activity, comprising, screening a cDNA or mRNA library generated from a senescent or near-senescent cell for members that suppress telomerase activity in an embryonic stem cell.

48. The method of claim 47 whereby a decrease in telomerase activity is measured by measuring for decreased expression of a telomerase reporter gene.

49. The method of claim 47 wherein said telomerase reporter gene is a construct comprising the hTRT gene fused to a reporter gene.

50. The method of claim 49 wherein the construct comprises a gene fusion.

51. The method of claim 49 wherein the construct comprises a protein fusion.

52. The method of claim 47 whereby telomerase activity is decreased via a protein interaction, and a decrease in telomerase activity is measured via the TRAPeze assay.

53. The method of claim 47 whereby said cDNA or mRNA library is subjected to subtractive hybridization with a cDNA or mRNA library from an embryonic stem cell prior to library screening.

54. A method of identifying a protein that enhances telomerase activity, comprising
a. collecting fractions from the cytoplasm of an oocyte,
b. adding them to a cell-free system designed from a senescent or near-senescent cell, and
c. measuring for changes in telomerase activity that result from exposure to specific oocyte cytoplasmic fractions.

55. A gene identified by the method of claim 40.

56. A gene identified by the method of claim 47.

57. A protein identified by the method of claim 54.

58. A method for screening for compounds that inhibit telomerase activity, comprising exposing an embryonic stem cell generated by nuclear transfer techniques using a senescent or near-senescent donor cell to a compound to determine whether said compound inhibits telomerase activity.

59. A compound identified by the method of claim 58.

60. A pharmaceutical composition comprising the gene of claim 55, or a portion or a transcription product thereof, for the purpose of enhancing telomerase activity in a subject in need of such enhanced activity.

61. A pharmaceutical composition comprising the gene product encoded by the gene of claim 55 for the purpose of enhancing telomerase activity in a subject in need of such enhanced activity.

62. A pharmaceutical composition comprising the gene of claim 56, or a portion or a transcription product thereof, for the purpose of suppressing telomerase activity in a subject in need of such suppressed activity.

63. A pharmaceutical composition comprising the gene product encoded by the gene of claim 56 for the purpose of suppressing telomerase activity in a subject in need of such suppressed activity.

64. A pharmaceutical composition comprising the protein of claim 58 for the purpose of enhancing telomerase activity in a subject in need of such enhanced activity.

65. A gene encoding the protein of claim 58.

66. A pharmaceutical composition comprising the gene of claim 65 for the purpose of enhancing telomerase activity in a subject in need of such enhanced activity.

67. A pharmaceutical composition comprising the compound of claim 59 for the purpose of inhibiting telomerase activity in a patient in need of such decreased activity.

68. A method for activating endogenous telomerase for the purpose of extending the life span of a primary cell.
